(19) United States
(12) Patent Application Publication
(10) Pub. No.: US 2007/0027474 A1
(43) Pub. Date: Feb. 1, 2007

(54) Pressure limiting forceps

(76) Inventor: Jeffrey Lasner, Purchase, NY (US)

Correspondence Address: Alfred E. Miller, 445 West Putnam Avenue, Greenwich, CT, 06830, US

(21) Appl. No.: 11/486,942

(22) Filed: Jul. 17, 2006

Related U.S. Application Data
(60) Provisional application No. 60/699,574, filed on Jul. 15, 2005.

(51) Int. Cl. A61B 17/08

(52) U.S. Cl. 606/219

(57) ABSTRACT

Forceps of the tips that are commonly used in surgical procedures having pressure limiting means that is effective after the tissue or material is firmly grasped by the platforms or tips, thus causing pressure being applied and competent damage to the tissue or material being handled by the forceps.

### PRIORITY CLAIM

This invention was first filed as a U.S. provisional Application No. 60/699,574 on Jul. 15, 2005. The filing date and priority of this first filing is expressly claimed pursuant to 35 U.S.C. § 119 (e).

### FIELD OF THE INVENTION

This invention relates generally to forceps used in a surgical procedure relating to tissue and grafts having a construction which limits or reduces the pressure on the surgeon's hand while doing said procedure but maintaining a secure grasp with minimal pressure.

### BACKGROUND OF THE INVENTION

In the surgical field, it is known that a problem exists in the handling of tissues and grafts with conventional forceps. When using currently available forceps the closing force is transmitted directly and proportionally to the tips of the forceps. As a consequence the surgeon needs to be aware of the pressure exerted on the tissue during manipulation. Too little pressure will result in slippage, and too much force applied to the forceps could result in tearing, puncturing, abrasion or scoring which is the primary reason for tissue breakdown and limited survivorship of surgical procedures, such as bypass and bowel resections.

When a surgeon uses a forceps it is generally held in the surgeon's non-dominant hand since the surgeon is normally concentrating and directing attention on what the dominant hand is doing so that it is clear that it is entirely possible to place an extra 20-30 grams of excess pressure on the very fragile tissue by means of the forceps. This excess pressure is transmitted directly to the tissue, thus causing significant trauma. This trauma causes the anatomic site to heal more slowly and break down over time, thus causing the ultimate failure of the surgery.

### SUMMARY OF THE INVENTION

The present invention has been developed in order to overcome the propensity of surgeons to inadvertently apply excess pressure on forceps during surgical procedures resulting in tissue damage. In this regard the forceps are provided with means for limiting or reducing the pressure exerted on tissue by an arrangement of stress relief that is built into the instrument. Thus, the pressure exerted on the forceps is automatically limited or reduced by means of a flexible resilient arms attached to the main forceps construction that is made of relatively strong and inflexible stainless steel, or any other surgical quality material.

Furthermore, in the preferred embodiment of the invention, additional stress relief is obtained by means of the flexible interlocking sleeves at the distal end of the forceps. It should be noted, however, that the present forceps instrument will provide stress relief with the flexible arm or arms alone connected between the main body and the gripping platforms of the forceps.

The above and other features of the present invention will be apparent by reference to the following description of my invention together with the accompanying drawings.

### BRIEF DESCRIPTION OF THE DRAWINGS

FIG. 1 is a perspective view of the pressure limiting forceps constructed in accordance with the teachings of my invention.

FIG. 1A is an enlarged partial perspective view of the forward end of the pressure limiting forceps shown in FIG. 1.

FIG. 2 is a side elevational view of the pressure limiting forceps in its natural inactive state.

FIG. 3 is another side elevational view of the pressure limiting forceps in an active state with the platforms of the forceps commencing to grasp tissue.

FIG. 4 is a further side elevational view of the forceps showing the handle of the forceps being further squeezed by the user's hands so that the tissue is firmly grasped at it's maximal pressure and

FIG. 5 is a top plan view of the pressure limiting forceps of the present invention.

### DESCRIPTION OF THE PREFERRED EMBODIMENT

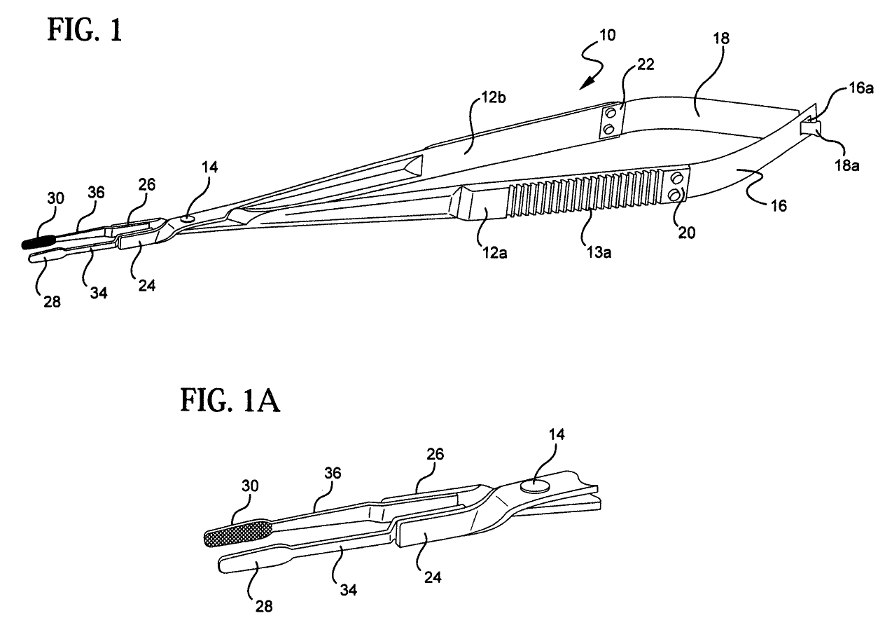

Forceps are presently used by health professionals, such as surgeons, in connection with procedures involving tissue manipulation. In order to ensure the proper amount of pressure is applied and controlled the present invention, as shown in FIGS. 1-3 of the drawings, has been devised in which the pressure limiting forceps is shown generally by the numeral 10. The main body of the forceps includes a handle that is provided with two parts 12a and 12b respectively which are pivotally connected at the forward end of the forceps by means of pivot 14. The handles 12a and 12b are provided with slip resistant grasping surfaces 13a and 13b. The distal end of the forceps has flexible interlocking bands 16 and 18 each being attached at to the handles 12a and 12b by securing means 20 and 22. The other ends of the flexible bands 16 and 18 are male-female connected by means of projection 18a of band 18 free sliding in opening 16a of the band 16.

As seen in FIG. 1A, the forward end of the forceps in front of the pivot 14 have stops 24 and 26 made of strong and inflexible material. The gripping platforms or tips 28 and 30 are also fabricated of strong and inflexible material, such as stainless steel or any other surgical quality material. Furthermore, it is preferable to provide the gripping platforms 28 and 30 with a slip resistant surface, such as carbide interdigitating serrated tips 32.

Connected to the main body of the forceps are resilient arms 34 and 36 that are attached to the inside of stops 24 and 26. The arms 34 and 36 are constructed of highly flexible material with a high modulus of elasticity and low deformation probability.

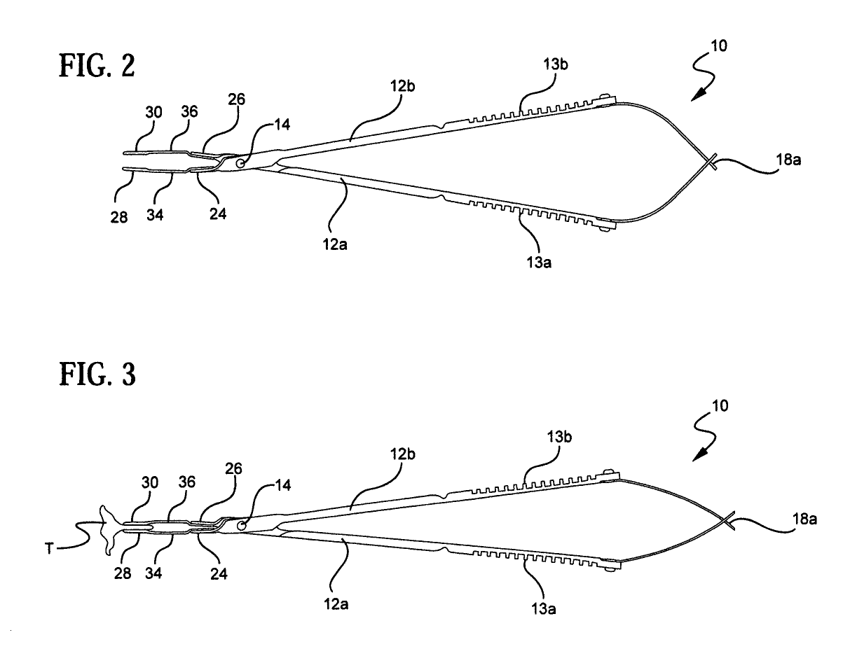

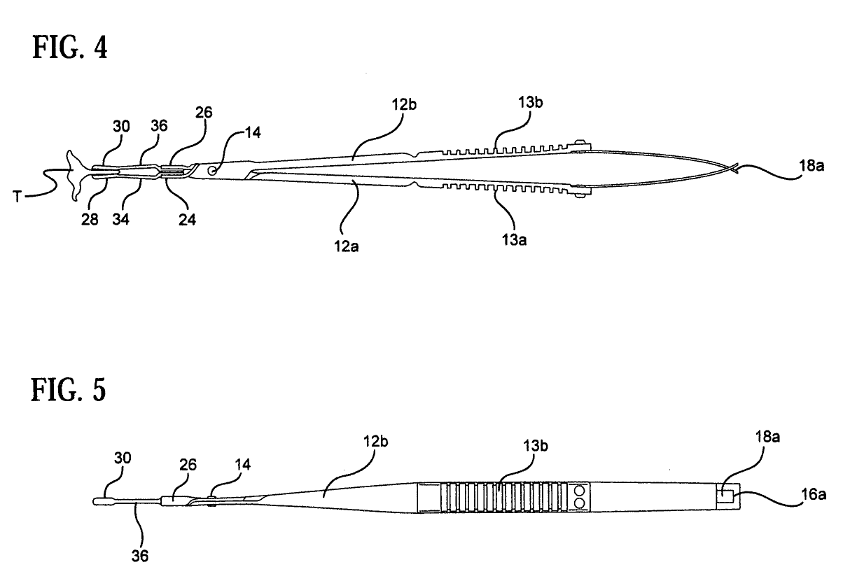

Referring now to FIGS. 2-4, when a surgeon is using the pressure limiting forceps in his or her non-dominant hand during a surgical procedure while the dominant hand has a surgical instrument for carrying out the particular procedure, extreme care must be exercised on the amount of pressure exerted on the forceps grasping delicate tissue. With the present forceps in which the handle parts 12a and 12b are squeezed toward one another the tissue is grasped, as seen in FIG. 3. As seen in FIG. 4, further pressure is applied to the handles 12a and 12b when the handles are being squeezed closer together, and the gripping platforms 28 and 30 apply more pressure on the firmly grasped tissue continuously to a point just prior to complete forceps closure. At this point the stops 24 and 26 become effective through the connected ends of resilient arms 34 and 36.

When a stop arrangement is effected as seen as FIG. 4 the platforms of the forceps, which are firmly holding the tissue presented from further closure since arms 34 and 36 flex to relieve the excess pressure. In this case, the long axis of each of the arms 34 and 36 flex to the said point of complete forceps closure and continues to flex up to and including complete closure. When the platforms 28 and 30 are completely closed any additional pressure applied to the forceps results in stress relief by means of the flexible resilient arms 34 and 36, as well as by the flexible interlocking bands 16 and 18. Thus, the present arrangement results in a net pressure release on the tissue T. Excess pressure on the forceps is thus relieved on both the front and distal ends of the forceps.

Although the present described forceps is shown handling tissue in a surgical procedure, it should be apparent that the forceps can be used also wherever delicate material in any discipline must be handled to avoid tearing, puncturing or marring of the delicate or fragile material.

While the present invention has been disclosed and described with reference to a single embodiment, it will be apparent that changes and modifications may be made therein, and it is intended in the following claims to cover each variation and modification as falls within the true spirit and scope of the invention.

What is claimed is:

1. Pressure limiting forceps for handling delicate material having a main body including a handle, flexible, interlocking male-female end bonds connected to said main body at the distal end of said forceps, a pair of gripping platforms at the forward end of said forceps, and at least one flexible resilient arm connecting one of said platforms to said main body whereby when manual pressure is applied to said platforms to grip said delicate material to a point where there is complete closure of the platforms on said material and further pressure on said platforms results in pressure release on the material by the flexing of the resilient arm and the flexing of the interlocking end bands.

2. Pressure limiting surgical forceps for handling tissue having a main body including a handle, flexible interlocking male-female end bands connected to said main body at the distal end of said forceps, a pair of flexible resilient arms each connecting a platform to said main body whereby when manual pressure is applied to said platforms to grip said tissue to a point where there is complete closure of said platforms additional manual pressure results in pressure relief on said tissue by the flexing of the resilient arms and the additional flexing of the interlocking end bands.

3. The pressure limiting surgical forceps as claimed in claim 2 wherein said resilient arms are fabricated of a highly flexible material with a high modulus of elasticity and a low deformation probability.

4. The pressure limiting surgical forceps as claimed in claim 2 wherein said main body is fabricated of inflexible stainless steel or other surgical quality material.

5. The pressure limiting surgical forceps as claimed in claim 2 further comprising stop elements on the forward end of said main body and the ends of said resilient arms remote from said platforms being affixed to the inside of said stop elements.

6. The pressure limiting surgical forceps as claimed in claim 2 wherein said handle is provided with slip resistant grasping surfaces.

7. The pressure limiting surgical forceps as calmed in claim 2 wherein the gripping platforms are provided with a slip resistant surface.

8. The pressure limiting procedure as claimed in claim 7 wherein said slip resistant surfaces are carbide interdigitating serrated tips.

9. Pressure limiting surgical forceps for handling tissue comprising a main body having a handle, flexible interlocking and slidable male-female end bands connected to said main body at the distal end of said forceps, stop members at the forward end of said main body, a pair of gripping platforms at the forward end of said forceps, a pair of flexible resilient arms each connecting a platform to said main body whereby when manual pressure is applied to said platforms to grip said tissue to a point where the stops become operative and there is complete closure of the platforms on said tissue whereby further pressure release on said tissue is caused by the flexing of said resilient arms and the additional flexing of the interlocking end bands.

10. Pressure limiting forceps for handling tissue comprising a main body having a handle, a pair of gripping tips at the forward end of said forceps, stop members at the forward end of said forceps, stops, stop members at the forward end of said main body, at least one flexible resilient arms connecting one of said tips to said main body whereby when manual pressure is applied to said tips to grip said tissue to a point where the stops become operational and there is complete closure exerted on said tips results in flexing of said arm and pressure relief.
